(19) United States
(12) Patent Application Publication
(10) Pub. No.: US 2007/0248665 A1
(43) Pub. Date: Oct. 25, 2007

(54) Compositions comprising co-precipitate of eplerenone and a water-soluble excipient

(76) Inventor: Bernard Charles Sherman, Toronto (CA)

Correspondence Address: NIXON & VANDERHYE, PC, 901 NORTH GLEBE ROAD, 11TH FLOOR, ARLINGTON, VA, 22203, US

(21) Appl. No.: 11/409,270

(22) Filed: Apr. 24, 2006

(51) Int. Cl. A61K 31/58; A61K 9/20

(52) U.S. Cl. 424/464; 514/173

(57) ABSTRACT

Solid compositions for oral administration comprising a co-precipitate of eplerenone and a water-soluble excipient.

### BACKGROUND OF THE INVENTION

Eplerenone is an aldosterone antagonist. It can be administered to treat pathological conditions associated with hyperaldosteronism such as hypertension, cardiac insufficiency and cirrhosis of the liver. Tablets comprising eplerenone are sold in the United States and elsewhere under the tradename Inspra™ in strengths of 25 mg and 50 mg.
™ Trademark.

U.S. patent application Ser. No. 09/456,614 filed on Dec. 8, 1999, issued to U.S. Pat. No. 6,410,054 on Jun. 25, 2002, relates to immediate release eplerenone compositions, and it appears that Inspra™ tablets are made according to the teachings of this publication. U.S. Pat. Nos. 6,495,165, 6,534,093, 6,558,707, 6,592,902, and 6,863,902 were all issued from this patent application or divisional applications therefrom.

As explained in U.S. patent application Ser. No. 09/456,614, now U.S. Pat. No. 6,410,054, eplerenone's low solubility in water makes it difficult to produce compositions from which bioavailability is adequate on oral administration. This publication teaches that the problem can be overcome by using particulate eplerenone micronized to small particle size. Accordingly, the claims of the patents issued from that application and its divisionals cover compositions comprising particulate eplerenone in micronized form, which enable rapid dissolution in gastrointestinal fluid, and consequently improved bioavailability.

The cost of micronization adds to the cost of the production; and additionally, there are significant losses of material in the micronization process.

It is thus the objective of the present invention to enable eplerenone compositions that exhibit rapid dissolution in aqueous media, and thus improved bioavailability, without the need for micronization of the eplerenone.

### DESCRIPTION OF THE INVENTION

It has been found that the rate of dissolution of eplerenone in aqueous media can be substantially increased, without micronization, by use of a co-precipitate of eplerenone and a water-soluble excipient. A “co-precipitate” will be understood to mean a solid substance that results from dissolving eplerenone together with the water-soluble excipient in volatile solvent and evaporating the solvent. The co-precipitate is not particulate eplerenone, but is comprised of molecules of eplerenone interspersed with molecules of the water-soluble excipient. The effect of the interspersed molecules of water-soluble excipient is to substantially increase the dissolution rate in aqueous media.

The water-soluble excipient will preferably be a polymer, and will most preferably be povidone.

The volatile organic solvent will preferably be or comprise a chlorinated hydrocarbon, most preferably methylene chloride.

The co-precipitate can be made, for example, by dissolving the eplerenone and water-soluble excipient in volatile organic solvent and spray-drying the solution. The resulting co-precipitate can then be mixed with other excipients, and the mixture compressed into tablets.

Alternatively, instead of spray-drying the solution, the solution can be sprayed onto other excipients in a fluidized-bed dryer. The dried mixture can then be mixed with other excipients, and the mixture compressed into tablets.

Alternatively, the solution of eplerenone and water-soluble excipient in volatile organic solvent can be added to other excipients to form a wet mass, and the wet mass can then be dried, and the dried mixture can then be mixed with other excipients, and the mixture can then be compressed into tablets.

Alternatively, and most preferably, the solution of eplerenone and water-soluble excipient in volatile organic solvent can be gradually added to other excipients while mixing in a heated mixer. If the temperature is maintained at above the boiling point of the solvent, the solvent will be evaporated as the solution is added. This process can be most readily carried out in a jacketed mixer, heated by circulating a hot liquid throughout the jacket, most conveniently hot water. After the addition of the solution is complete, the dry mixture can be mixed with other excipients, and the mixture compressed into tablets.

The invention will be better understood from the following examples.

### EXAMPLE 1

75.0 g of eplerenone and 37.5 g of povidone were dissolved in about 2000 g of methylene chloride.

The solution was slowly added to 112.5 g microcrystalline cellulose and 75.0 g croscarmellose sodium while mixing in a jacketed mixer, while circulating hot water through the jacket, to maintain the temperature of the contents of the mixture at about 50° C.

At the end of this process, the dried mixture comprised 75.0 g eplerenone in a total drug weight of 300 g. The eplerenone content was thus 25% by weight.

### EXAMPLE 2

1.0 g magnesium stearate (as lubricant) was mixed with 100 g of the product from example 1, and the mixture was compressed into tablets, at a tablet weight of 202 mg. Each tablet thus contained 50 mg eplerenone.

Tablets of this example were tested for dissolution rate in 900 mL 0.1N HCl in USP apparatus 2 at 50 rpm. It was found that over 80% dissolved in 20 minutes and that the dissolution profile was virtually superimposable to that of Inspra™ tablets.

1. A solid pharmaceutical composition comprising a co-precipitate of eplerenone and a water-soluble excipient.

2. A composition of claim 1 wherein the water-soluble excipient is a polymer.

3. A composition of claim 2 wherein the polymer is providone.

4. A composition of claim 1 in the form of a tablet.

5. A composition of claim 1, wherein the co-precipitate is made by the dissolving eplerenone and a water-soluble excipient in volatile organic solvent and evaporating the solvent.

6. A composition of claim 5, wherein the volatile organic solvent is or comprises a chlorinated hydrocarbon.

7. A composition of claim 6, wherein the chlorinated hydrocarbon is or comprises methylene chloride.

8. A composition of claim 1 made by a process wherever a solution of eplerenone and a water-soluble excipient in volatile organic solvent is spray-dried.

9. A composition of claim 1 made by a process wherein a solution of eplerenone and a water-soluble excipient in a volatile organic solvent is sprayed onto other excipients in a fluid bed dryer.

10. A composition of claim 1 made by a process wherein a solution of eplerenone and a water-soluble excipient in a volatile organic solvent is added to other excipients in a mixer, and the wet mass is then dried to evaporate the solvent.

11. A composition of claim 1 made by a process wherein a solution of eplerenone and water-soluble excipient in volatile organic solution is added to other excipients in a mixer that is heated so as to evaporate solvent as the solution is added.
